(19) United States
(12) Patent Application Publication
(10) Pub. No.: US 2025/0372242 A1
(43) Pub. Date: Dec. 4, 2025

(54) INFORMATION PROCESSING APPARATUS, INFORMATION PROCESSING METHOD, AND NON-TRANSITORY COMPUTER READABLE MEDIUM

(71) Applicant: NEC Corporation, Tokyo (JP)

(72) Inventors: Takamu MATSUMOTO, Tokyo (JP); Manami HOSAKA, Tokyo (JP); Jun TAKIKAWA, Tokyo (JP)

(73) Assignee: NEC Corporation, Tokyo (JP)

(21) Appl. No.: 19/207,534

(22) Filed: May 14, 2025

(30) Foreign Application Priority Data
May 28, 2024 (JP) 2024-086516

(51) Int. Cl. G16H 40/20; G16H 10/60

(52) U.S. Cl. CPC G16H 40/20; G16H 10/60

(57) ABSTRACT

The information processing apparatus includes at least one memory storing instructions and at least one processor that executes the instructions. The at least one processor executes instructions to acquire index information including a plurality of indexes related to a target medical institution, acquire an analysis result output by an analysis model that that has referred to at least a part of the index information, acquire explanatory information regarding at least a part of the analysis result, the explanatory information being information output from a generation model that has been subjected to machine learning and has referred to at least a part of the analysis result, and generate output data including at least a part of the analysis result and at least a part of the explanatory information.

### INCORPORATION BY REFERENCE

This application is based upon and claims the benefit of priority from Japanese patent application No. 2024-086516, filed on May 28, 2024, the disclosure of which is incorporated herein in its entirety by reference.

### TECHNICAL FIELD

The present disclosure relates to an information processing apparatus, an information processing method, and a non-transitory computer readable medium.

### BACKGROUND ART

A technique for supporting management of a medical institution or the like is known. For example, Japanese Unexamined Patent Application Publication No. 2010-218448 discloses a hospital management evaluation support system that performs cost calculation per labor cost on the basis of revenue data and expense data and outputs hospital management evaluation data including a relationship between RMP and hospital management index data.

### SUMMARY

Analysis results using a system as disclosed in Japanese Unexamined Patent Application Publication No. 2010-218448 have high expertise, and it is often difficult for a person who is not an expert to understand the analysis results or it takes a long time to understand the analysis results.

The present disclosure has been made in view of the above problems, and an example object thereof is to provide a technique for presenting an analysis result regarding an index of a medical institution in an easily understandable manner. An example object of the present disclosure is to provide an information processing apparatus, an information processing method, and a non-transitory computer readable medium storing a program.

An information processing apparatus according to a first example aspect of the present disclosure includes first acquisition means for acquiring index information including a plurality of indexes related to a target medical institution; second acquisition means for acquiring an analysis result output by an analysis model that has referred to at least a part of the index information; third acquisition means for acquiring explanatory information regarding at least a part of the analysis result, the explanatory information being information output from a generation model that has been subjected to machine learning and has referred to at least a part of the analysis result; and generation means for generating output data including at least a part of the analysis result and at least a part of the explanatory information.

An information processing method according to a second example aspect of the present disclosure includes acquiring index information including a plurality of indexes related to a target medical institution; acquiring an analysis result output by an analysis model that has referred to at least a part of the index information; acquiring explanatory information regarding at least a part of the analysis result, the explanatory information being information output from a generation model that has been subjected to machine learning and has referred to at least a part of the analysis result; and generating output data including at least a part of the analysis result and at least a part of the explanatory information.

A program according to a third example aspect of the present disclosure is a program that causes a computer to function as an information processing apparatus, the program causing the computer to function as first acquisition means for acquiring index information including a plurality of indexes related to a target medical institution; second acquisition means for acquiring an analysis result output by an analysis model that has referred to at least a part of the index information; third acquisition means for acquiring explanatory information regarding at least a part of the analysis result, the explanatory information being information output from a generation model that has been subjected to machine learning and has referred to at least a part of the analysis result; and generation means for generating output data including at least a part of the analysis result and at least a part of the explanatory information.

According to an example aspect of the present disclosure, it is possible to provide a technique for presenting an analysis result related to an index of a medical institution in an easily understandable manner.

### BRIEF DESCRIPTION OF DRAWINGS

The above and other aspects, features and advantages of the present disclosure will become more apparent from the following description of certain exemplary embodiments when taken in conjunction with the accompanying drawings, in which:

FIG. 1 is a block diagram showing a configuration of an information processing apparatus according to the present disclosure;

FIG. 2 is a flowchart showing a flow of an information processing method according to the present disclosure;

FIG. 3 is a block diagram showing a configuration of the information processing apparatus according to the present disclosure;

FIG. 4 is a sequence diagram showing a flow of processing in an information processing system according to the present disclosure;

FIG. 5 is a diagram for describing information processing according to the present disclosure;

FIG. 6 is a diagram for describing information processing according to the present disclosure;

FIG. 7 is a diagram for describing information processing according to the present disclosure;

FIG. 8 is a diagram for describing information processing according to the present disclosure;

FIG. 9 is a diagram for describing information processing according to the present disclosure; and

FIG. 10 is a block diagram showing a configuration of a computer that functions as the information processing apparatus according to the present disclosure.

### EMBODIMENTS

Hereinafter, example embodiments of the present disclosure will be described. However, the present disclosure is not limited to the exemplary embodiments described below, and various modifications can be made within the scope described in the claims. For example, example embodiments obtained by combining the techniques (some or all of the products or methods) adopted in the following exemplary embodiments as appropriate can also be included in the scope of the present disclosure. Example embodiments obtained by omitting some of the techniques adopted in the following exemplary embodiments as appropriate can also be included in the scope of the present disclosure. The effects mentioned in the following exemplary embodiments are examples of effects expected in the exemplary embodiments, and do not define the extension of the present disclosure. That is, example embodiments that do not achieve the effects mentioned in the following exemplary embodiments can also be included in the scope of the present disclosure.

### First Example Embodiment

A first exemplary embodiment, which is an example embodiment of the present disclosure, will be described in detail with reference to the drawings. The present exemplary embodiment is a basic form of each exemplary embodiment described below. Note that the application scope of each technique adopted in the present exemplary embodiment is not limited to the present exemplary embodiment. That is, each technique adopted in the present exemplary embodiment can also be adopted in other exemplary embodiments included in the present disclosure as long as no particular technical problem occurs. Each technique shown in the drawings referred to for describing the present exemplary embodiment can also be adopted in other exemplary embodiments included in the present disclosure as long as no particular technical problem occurs.

### (Configuration of Information Processing Apparatus 1)

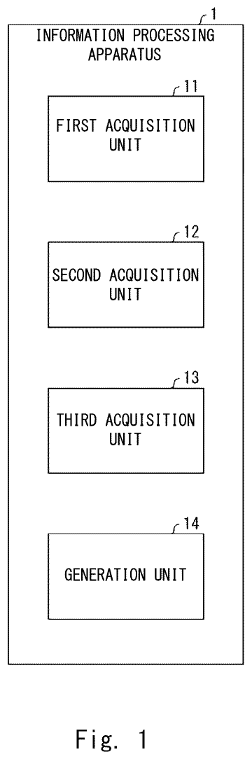

A configuration of an information processing apparatus 1 according to the present exemplary embodiment will be described with reference to FIG. 1. FIG. 1 is a block diagram showing a configuration of the information processing apparatus 1. As shown in FIG. 1, the information processing apparatus 1 includes a first acquisition unit 11, a second acquisition unit 12, a third acquisition unit 13, and a generation unit 14.

### (First Acquisition Unit 11)

The first acquisition unit 11 acquires index information including a plurality of indexes related to a target medical institution. Here, the target medical institution may be one medical institution or a plurality of medical institutions. Also, medical institutions may include, for example, hospitals, clinics, midwifery homes, nursing homes, home nursing stations, and pharmacies, although these examples are not intended to limit the present exemplary embodiments.

The “index” is not particularly limited as long as it is data that can be analyzed by an analysis model that will be described later, but as an example, may include an index related to the management of a medical institution (also referred to as a management index). The “index” may include data regarding medical care fees, data included in an electronic medical chart, other reference information, and the like, but these examples also do not limit the present exemplary embodiment.

### (Second Acquisition Unit 12)

The second acquisition unit 12 acquires an analysis result output by an analysis model that refers to at least a part of the index information acquired by the first acquisition unit 11. As an example, the second acquisition unit 12 is configured to perform processes of
generating input data to be input to the analysis model with reference to the index information,
inputting the generated input data to the analysis model, and
acquiring an analysis result output by the analysis model.
   Alternatively, the second acquisition unit 12 may be expressed as generating an analysis result by inputting at least a part of the index information acquired by the first acquisition unit 11 to the analysis model. Here, the analysis model may be included in the second acquisition unit 12 or the information processing apparatus 1, or may be included in a server apparatus or the like outside the information processing apparatus 1. An analysis model subjected to machine learning may be used as the analysis model. Note that the specific processing content of the above analysis model does not limit the present exemplary embodiment, but may include, for example,
data pre-processing,
feature amount design, and
post processing of data.
   Here, the feature amount design may include respective processes such as
construction of a feature amount space,
extraction of a feature amount, and
verification of a feature amount.
   The verification of a feature amount may include each process such as
verification of a correlation between a plurality of feature amounts. In the case of such a configuration, the analysis result may include, for example, a plurality of feature amounts extracted by the analysis model and information regarding a correlation between the plurality of feature amounts. Here, the plurality of feature amounts may include
any of a plurality of indexes included in the index information described above, or
an index obtained by combining a plurality of indexes included in the above-described index information.
   Therefore, the analysis result may be expressed as including information regarding a correlation between
one or a plurality of indexes obtained from the index information, the one or plurality of indexes serving as a target index that is an analysis target, and
one or a plurality of indexes obtained from the index information, the one or plurality of indexes serving as a factor index that is a factor of the target index.

### (Third Acquisition Unit 13)

The third acquisition unit 13 acquires explanatory information regarding at least a part of the analysis result, the explanatory information being output from a generation model that has been subjected to machine learning and has referred to at least a part of the analysis result acquired by the second acquisition unit 12. As an example, the third acquisition unit 13 may be configured to perform processes of
generating a prompt with reference to a part of the analysis results,
inputting the generated prompt to the generation model, and
acquiring a generation result generated by the generation model.
   Alternatively, the third acquisition unit 13 may be expressed as generating the explanatory information by inputting at least a part of the analysis result acquired by the second acquisition unit 12 to the generation model subjected to machine learning. Here, the generation model may be included in the third acquisition unit 13 or the information processing apparatus 1, or may be included in a server apparatus or the like outside the information processing apparatus 1. A large language model subjected to machine learning may be used as the generation model.

The generation result generated by the generation model may include at least one of, for example,
explanatory information for explaining content of at least a part of the analysis result, and
a prediction model for performing a prediction process based on the analysis result.

Note that the specific example of the explanatory information does not limit the present exemplary embodiment, but the explanatory information includes, as an example, information for explaining one or a plurality of feature amounts (in other words, the target index or the factor index) included in the analysis result. The explanatory information may include, for example,
more specific definition regarding one or a plurality of feature amounts included in information/analysis results for explaining one or a plurality of feature amounts included in the analysis result in a more easily understandable manner.

### (Generation Unit 14)

The generation unit 14 generates output data including at least a part of the analysis result acquired by the second acquisition unit 12 and at least a part of the explanatory information acquired by the third acquisition unit 13. The generated output data is presented to a user via an input/output unit (not shown) or the like as an example.

Note that, as an example, the user may include a person related to the target medical institution (a management executive, an accountant, or medical personnel (a doctor, a nurse, or the like)), or may include an administrator (operator) of the information processing apparatus 1.

### (Effects of Information Processing Apparatus 1)

As described above, the information processing apparatus 1 is configured to
acquire index information including a plurality of indexes related to a target medical institution,
acquire an analysis result output by an analysis model that has referred to at least a part of the index information,
acquire explanatory information that is output by generation model that has been subjected to machine learning and referred to at least a part of the analysis result, the explanatory information being information regarding at least a part of the analysis result, and
generate output data including at least a part of the analysis result and at least a part of the explanatory information.
   As described above, since the output data generated by the information processing apparatus 1 includes at least a part of the analysis result and at least a part of the explanatory information, it is possible to present the analysis result of the index related to the target medical institution to the user in an easily understandable manner.

### (Flow of Information Processing Method S1)

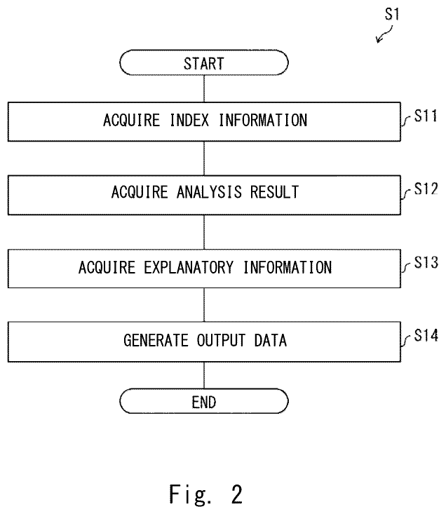

Next, a flow of an information processing method SI according to the present exemplary embodiment will be described with reference to FIG. 2. FIG. 2 is a flowchart showing a flow of the information processing method S1. As shown in FIG. 2, the information processing method S1 includes step (process) S11 of acquiring index information, a step (process) S12 of acquiring an analysis result, a step (process) S13 of acquiring explanatory information, and a step (process) S14 of generating output data.

### (Step S11)

In step S11, the first acquisition unit 11 acquires index information including a plurality of indexes related to a target medical institution. Since a more specific description of the first acquisition unit 11 has been described above, the description thereof will be omitted here.

### (Step S12)

In step S12, the second acquisition unit 12 acquires an analysis result output by an analysis model that has referred to at least a part of the index information acquired by the first acquisition unit 11. A more specific description of the second acquisition unit 12 has been described above, and thus description thereof will be omitted here.

### (Step S13)

In step S13, the third acquisition unit 13 acquires explanatory information regarding at least a part of the analysis result, the explanatory information being output from a generation model that has been subjected to machine learning and has referred to at least a part of the analysis result acquired by the second acquisition unit 12. A more specific description of the third acquisition unit 13 has been described above, and thus description thereof will be omitted here.

### (Step S14)

In step S14, the generation unit 14 generates output data including at least a part of the analysis result acquired by the second acquisition unit 12 and at least a part of the explanatory information acquired by the third acquisition unit 13. Since a more specific description of the generation unit 14 has been described above, the description thereof will be omitted here.

### (Effect of Information Processing Method S1)

As described above, the information processing method S1 includes
acquiring index information including a plurality of indexes related to a target medical institution,
acquiring an analysis result output by an analysis model that has referred to at least a part of the index information,
acquiring explanatory information regarding at least a part of the analysis result, the explanatory information being output from a generation model that has been subjected to machine learning and has referred to at least a part of the analysis result, and
generating output data including at least a part of the analysis result and at least a part of the explanatory information.
   According to the above configuration, effects similar to those of the information processing apparatus 1 are obtained.

### Second Example Embodiment

A second exemplary embodiment, which is an example embodiment of the present disclosure, will be described in detail with reference to the drawings. Components having the same functions as the components described in the above-described exemplary embodiments are denoted by the same reference numerals, and the description thereof will be appropriately omitted. Note that the application scope of each technique adopted in the present exemplary embodiment is not limited to the present exemplary embodiment. That is, each technique adopted in the present exemplary embodiment can also be adopted in other exemplary embodiments included in the present disclosure as long as no particular technical problem occurs. Each technique shown in each drawing referred to for describing the present exemplary embodiment can also be adopted in other exemplary embodiments included in the present disclosure as long as no particular technical problem occurs.

### (Configuration of Information Processing System 1A)

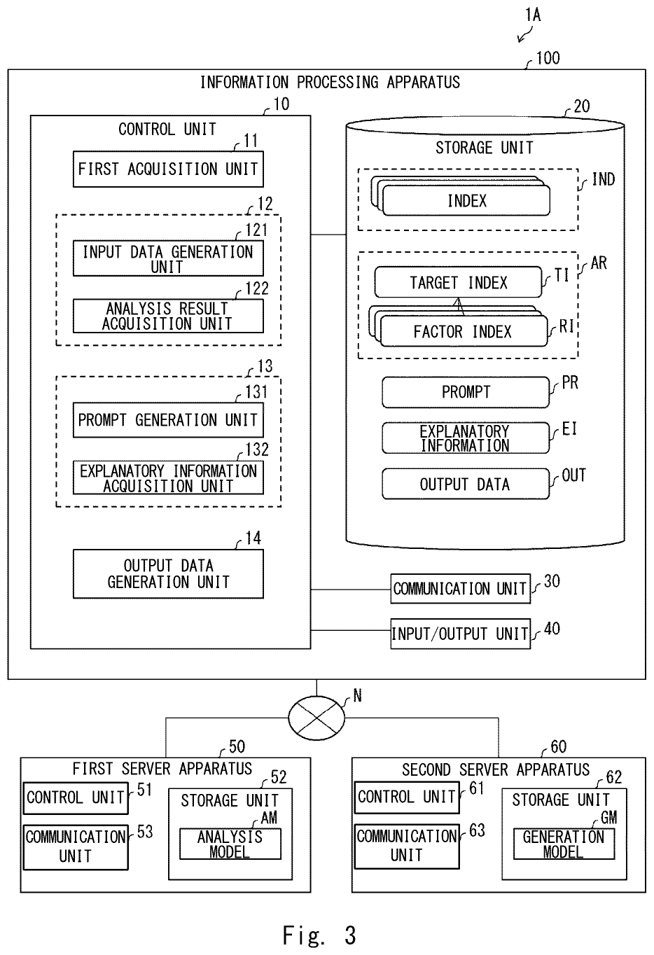

A configuration of an information processing system 1A according to the present exemplary embodiment will be described with reference to FIG. 3. FIG. 3 is a block diagram showing a configuration of the information processing system 1A. As shown in FIG. 3, the information processing system 1A includes an information processing apparatus 100, and a first server apparatus 50 and a second server apparatus 60 connected to the information processing apparatus 100 via a network N. Here, a specific configuration of the network N is not limited to the present exemplary embodiment, but as an example, a wireless local area network (LAN), a wired LAN, a wide area network (WAN), a public line network, a mobile data communication network, or a combination of these networks may be used.

### (First Server Apparatus 50)

As shown in FIG. 3, the first server apparatus 50 includes a control unit 51, a storage unit 52, and a communication unit 53. The communication unit 53 communicates with an apparatus outside the first server apparatus 50. As an example, the communication unit 53 communicates with the information processing apparatus 100 included in the information processing system 1A. The communication unit 53 transmits data supplied from the control unit 51 to the information processing apparatus 100, and supplies data received from the information processing apparatus 100 to the control unit 51. Note that the data received by the communication unit 53 from the information processing apparatus 100 may include input data generated by the information processing apparatus 100. The data provided by the communication unit 53 to the information processing apparatus 100 may include a result (analysis result) of an analysis model AM that will be described later having analyzed the input data.

The storage unit 52 stores the analysis model AM. As an example, the storage unit 52 stores a plurality of parameters defining the analysis model AM. These parameters are, as an example, parameters learned in advance through machine learning (parameters subjected to update processing through machine learning), but this does not limit the present exemplary embodiment.

The control unit 51 acquires an analysis result from the analysis model AM by using the analysis model AM. As an example, the control unit 51 inputs, to the analysis model AM, input data received from the information processing apparatus 100 and including the index information acquired by the first acquisition unit 11 described above, and acquires an analysis result of the input data generated by the analysis model AM. The analysis result is provided to the information processing apparatus 100 via the communication unit 53. Specific processing of the analysis model AM will be described later.

### (Second Server Apparatus 60)

As shown in FIG. 3, the second server apparatus 60 includes a control unit 61, a storage unit 62, and a communication unit 63. The communication unit 63 communicates with an apparatus outside the second server apparatus 60. As an example, the communication unit 63 communicates with the information processing apparatus 100 included in the information processing system 1A. The communication unit 63 transmits data supplied from the control unit 61 to the information processing apparatus 100, and supplies data received from the information processing apparatus 100 to the control unit 61. Note that the data received by the communication unit 63 from the information processing apparatus 100 may include a prompt generated by the information processing apparatus 100. The data provided by the communication unit 53 to the information processing apparatus 100 may include a generation result generated by a generation model GM that will be described later on the basis of the prompt.

The generation model GM is stored in the storage unit 62. As an example, the storage unit 62 stores a plurality of parameters defining the generation model GM. These parameters are, as an example, parameters learned in advance through machine learning (parameters subjected to update processing through machine learning), but this does not limit the present exemplary embodiment. As the generation model GM, a large language model subjected to machine learning may be used.

The control unit 61 acquires information generated by the generation model GM by using the generation model GM. As an example, the control unit 61 acquires the explanatory information generated by the generation model GM on the basis of the prompt received from the information processing apparatus 100 and including the analysis result from the analysis model AM described above. The explanatory information is provided to the information processing apparatus 100 via the communication unit 63. Specific processing of the generation model GM will be described later. Note that, in the present exemplary embodiment, the first server apparatus 50 and the second server apparatus 60 are shown as apparatuses separate from the information processing apparatus 100, but this does not limit the present exemplary embodiment. The control unit of the information processing apparatus 100 may have a function as the control unit 51 included in the first server apparatus 50 or an analysis model execution unit in the control unit 51. The control unit of the information processing apparatus 100 may have a function as a generation model execution unit in the control unit 61 or the control unit 61 included in the second server apparatus 60. Similarly, the analysis model AM stored in the storage unit 52 included in the first server apparatus 50 may be stored in the storage unit of the information processing apparatus 100, and the analysis model AM may be executed by the information processing apparatus 100 itself. The generation model GM stored in the storage unit 62 included in the second server apparatus 60 may be stored in the storage unit of the information processing apparatus 100, and the generation model GM may be executable by the information processing apparatus 100 itself.

### (Configuration of Information Processing Apparatus 100)

Next, a configuration of the information processing apparatus 100 according to the present exemplary embodiment will be described with reference to FIG. 3. As shown in FIG. 3, the information processing apparatus 100 includes a control unit 10, a storage unit 20, a communication unit 30, and an input/output unit 40.

### (Communication Unit 30)

The communication unit 30 communicates with an apparatus outside the information processing apparatus 100. As an example, the communication unit 30 communicates with the first server apparatus 50 and the second server apparatus 60. The communication unit 30 transmits data supplied from the control unit 10 to the first server apparatus 50 and the second server apparatus 60, and supplies data received from the first server apparatus 50 and the second server apparatus 60 to the control unit 10. Note that the data transmitted from the communication unit 30 to the first server apparatus 50 may include input data generated by an input data generation unit 121 that will be described later. The data transmitted from the communication unit 30 to the second server apparatus 60 may include a prompt generated by a prompt generation unit 131 that will be described later. The data received by the communication unit 30 from the first server apparatus 50 may include an analysis result of the input data by the analysis model AM. The data received by the communication unit 30 from the second server apparatus 60 may include a generation result based on the prompt by the generation model GM.

### (Input/Output Unit 40)

The input/output unit 40 includes at least one of input/output apparatuses such as a keyboard, a mouse, a display, a printer, and a touch panel. Alternatively, input/output apparatuses such as a keyboard, a mouse, a display, a printer, and a touch panel may be connected to the input/output unit 40. In the case of this configuration, the input/output unit 40 receives inputs of various types of information to the information processing apparatus 100 from the connected input apparatus. The input/output unit 40 outputs various types of information to the connected output apparatus under the control of the control unit 10. The input/output unit 40 may include an interface such as a Universal Serial Bus (USB).

### (Storage Unit 20)

The storage unit 20 stores various data referred to by the control unit 10 and various data generated by the control unit 10. The storage unit 20 stores, for example,
index information IND,
an analysis result AR,
a prompt PR,
explanatory information EI, and
output data OUT.
   Here, the index information IND includes a plurality of indexes related to one or a plurality of target medical institutions. The index information IND may include, for example,
an index related to management of a target medical institution (also referred to as a management index),
data regarding medical care fees at a target medical institution,
data included in an electronic medical chart in a target medical institution, and
other references in the target medical institution.
   Here, the management index may include indexes such as an average number of days in hospital, a hospital bed utilization rate, and the number of introduced patients, but the present exemplary embodiment is not limited to these examples.

The data regarding the medical care fees may be a part of diagnosis procedure combination (DPC) data as an example. The reference information may include various types of information that can be acquired in a medical office system or various department systems in a target medical institution.

As mentioned in the first exemplary embodiment, the medical institution may include, for example, a hospital, a clinic, a midwifery center, a nursing home, a home visit nursing station, and a pharmacy, but these examples do not limit the present exemplary embodiment.

The analysis result AR is an analysis result output by the analysis model AM that has referred to at least a part of the index information IND, and includes, for example,
a target index TI that is one or a plurality of indexes obtained from the index information IND and is an analysis target,
a factor index RI that is one or a plurality of indexes obtained from the index information IND and is a factor of the target index TI, and
information regarding a correlation between the target index TI and the factor index RI.
   A specific example of the analysis result AR will be described later.

The prompt PR is a prompt generated by a prompt generation unit 131 included in the third acquisition unit 13, which will be described later, and is a prompt input to the generation model GM. A specific example of the prompt PR will be described later.

The explanatory information EI is explanatory information generated by the generation model GM and is explanatory information for explaining at least a part of the content of the analysis result AR. As also mentioned in the first exemplary embodiment, the explanatory information EI may include
information for explaining one or a plurality of feature amounts (indexes) included in the analysis result AR in a more easily understandable manner, and
more specific definitions and the like regarding one or a plurality of feature amounts (indexes) included in the analysis result AR.
   A specific example of the explanatory information EI will be described later.

The output data OUT is data generated by an output data generation unit 14 to be described later, and includes, as an example, at least a part of the analysis result AR and at least a part of the explanatory information EI. The output data OUT is visually presented to the user via the input/output unit 40 as an example. A specific example of the output data OUT will be described later.

### (Control Unit 10)

As shown in FIG. 3, the control unit 10 includes a first acquisition unit 11, a second acquisition unit 12, a third acquisition unit 13, and an output data generation unit 14.

### (First Acquisition Unit 11)

The first acquisition unit 11 acquires the index information IND including a plurality of indexes related to a target medical institution. Here, the target medical institution may be one medical institution or a plurality of medical institutions. For the first acquisition unit 11, redundant description of the already described content will be omitted.

### (Second Acquisition Unit 12)

The second acquisition unit 12 acquires the analysis result AR output from the analysis model AM that has referred to at least a part of the index information IND acquired by the first acquisition unit 11. As shown in FIG. 3, the second acquisition unit 12 includes an input data generation unit 121 and an analysis result acquisition unit 122.

The input data generation unit 121 refers to the index information and generates input data to be input to the analysis model AM. The input data generation unit 121 inputs the generated input data to the analysis model AM included in the first server apparatus 50 via the communication unit 30. The analysis result acquisition unit 122 acquires an analysis result output by the analysis model AM.

Note that the specific processing content by the analysis model AM does not limit the present exemplary embodiment, but may include, for example,
data pre-processing,
feature amount design, and
post-processing of data.
   Here, the feature amount design may include respective processes such as
construction of a feature amount space,
extraction of a feature amount, and
verification of a feature amount.
   The verification of a feature amount may include each process such as
verification of a correlation between a plurality of feature amounts. In the case of such a configuration, the analysis result AR may include, for example, a plurality of feature amounts extracted from the input data by the analysis model AM and information regarding a correlation between the plurality of feature amounts. Here, the plurality of feature amounts may include
any of a plurality of indexes included in the index information described above, or
an index obtained by combining a plurality of indexes included in the above-described index information. Therefore, the analysis result AR may be expressed as including information regarding a correlation between
the target index TI that is one or a plurality of indexes obtained from the index information IND and is an analysis target, and
the factor index RI that is one or a plurality of indexes obtained from the index information IND and is a factor of the target index TI.

Here, one or a plurality of target indexes TI may include at least one of
an index related to patient admission and discharge,
an index related to a status of a hospital bed, and
an index related to cost incurred with medical service for a patient.
   As a specific example, the one or plurality of target indexes TI may include at least one of an average number of days in hospital, a hospital bed utilization rate, and the number of introduced patients.

As an example, the one or plurality of factor indexes RI may be indexes related to at least one of doctor information, time information, hospitalization information, outpatient information, disease information, treatment information, patient information, and medical care information included in the index information IND.

Note that at least a part of the target index TI and the factor index RI described above may include an index designated by the user. In other words, the input data generation unit 121 may be configured to
determine at least a part of the target index TI and the factor index RI on the basis of an input from the user, and
instruct the analysis model AM to generate the analysis result AR including at least a part of the target index TI and the factor index RI determined on the basis of the input from the user.

After the analysis result acquisition unit 122 acquires an analysis result ARI based on first input data, the input data generation unit 121 may further execute post-processing (post-analysis) by referring to the analysis result AR1. As an example, the input data generation unit 121 may generate second input data based on a result of the post-processing and input the second input data to the analysis model AM again, and the analysis result acquisition unit 122 may acquire an analysis result AR2 based on the second input data.

### (Third Acquisition Unit 13)

The third acquisition unit 13 acquires the explanatory information EI regarding at least a part of the analysis result AR, the explanatory information EI being information output by the generation model GM that has been subjected to machine learning and has referred to at least a part of the analysis result AR acquired by the second acquisition unit 12. As shown in FIG. 3, the third acquisition unit 13 includes a prompt generation unit 131 and an explanatory information acquisition unit 132.

The prompt generation unit 131 generates a prompt PR with reference to a part of the analysis result AR. The prompt generation unit 131 inputs the generated prompt PR to the generation model GM included in the second server apparatus 60 via the communication unit 30. The explanatory information acquisition unit 132 acquires a generation result generated by the generation model GM and including the explanatory information EI regarding at least a part of the analysis result AR.

Note that the generation result generated by the generation model may include a prediction model for performing a prediction process based on the analysis result. The specific example of the explanatory information EI does not limit the present exemplary embodiment, but the explanatory information EI includes, as an example, information for explaining one or a plurality of feature amounts (in other words, the target index TI or the factor index RI) included in the analysis result AR. The explanatory information EI may include, for example,
information for explaining one or a plurality of feature amounts included in the analysis result AR in a more easily understandable manner, and
more specific definitions regarding one or a plurality of feature amounts included in the analysis result AR.

Note that the prompt generation unit 131 may generate the prompt PR to be input to the generation model GM by using a prompt template preset for each factor index RI. The prompt generation unit 131 may generate the prompt PR with further reference to an input from the user. As an example, the prompt generation unit 131 may be configured to
generate a first prompt PR1 by using a prompt template preset for each factor index RI,
present the first prompt PR1 to the user via the input/output unit 40,
generate a second prompt PR2 by modifying the first prompt PR1 on the basis of an input from the user, and
input the second prompt PR2 to the generation model GM.

### (Output Data Generation Unit 14)

The output data generation unit 14 generates output data including at least a part of the analysis result acquired by the second acquisition unit 12 and at least a part of the explanatory information acquired by the third acquisition unit 13. The generated output data is presented to the user via the input/output unit 40 or the like as an example. As an example, the output data includes information for supporting decision-making regarding management of the target medical institution.

### (Flow of Process S100 in Information Processing System 1A)

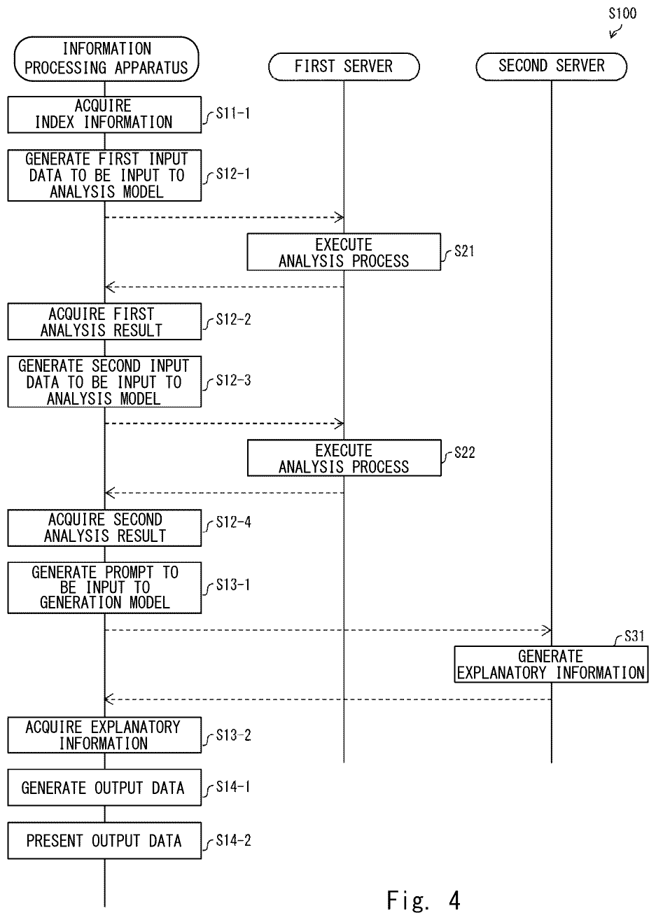

FIG. 4 is a sequence diagram showing an example of a flow of the process S100 in the information processing system 1A. Although FIG. 4 shows an example in which an analysis result in the analysis model AM included in the first server apparatus 50 is executed a plurality of times, this does not limit the present exemplary embodiment.

### (Step S11-1)

In step S11-1, the first acquisition unit 11 included in the information processing apparatus 100 acquires the index information IND.

### (Step S12-1)

Subsequently, in step S12-1, the input data generation unit 121 included in the information processing apparatus 100 refers to the index information IND to generate first input data to be input to the analysis model AM, the first input data including at least a part of the index information IND, and inputs the generated first input data to the analysis model AM via the communication unit 30.

### (Step S21)

In step S21, the analysis model AM executes analysis on a plurality of indexes included in the first input data, and provides the first analysis result AR1 to the information processing apparatus 100.

### (Step S12-2)

In step S12-2, the analysis result acquisition unit 122 included in the information processing apparatus 100 acquires the first analysis result AR1 output by the analysis model AM.

### (Step S12-3)

In step S12-3, the input data generation unit 121 executes post-processing with reference to the first analysis result AR1. The input data generation unit 121 generates second input data to be input to the analysis model AM, the second input data being based on a result of the post-processing, and inputs the generated second input data to the analysis model AM via the communication unit 30.

### (Step S22)

In step S22, the analysis model AM executes analysis on a plurality of indexes included in the second input data, and provides the second analysis result AR2 to the information processing apparatus 100.

### (Step S12-4)

In step S12-4, the analysis result acquisition unit 122 acquires the second analysis result AR2 output from the analysis model AM.

### (Step S13-1)

In step S13-1, the prompt generation unit 131 included in the information processing apparatus 100 refers to the second analysis result AR2 to generate the prompt PR to be input to the generation model GM, and inputs the generated prompt PR to the generation model GM via the communication unit 30.

### (Step S31)

In step S31, the generation model GM executes the prompt PR and provides the explanatory information EI to the information processing apparatus 100.

### (Step S13-2)

In step S13-2, the explanatory information acquisition unit 132 included in the information processing apparatus 100 acquires the explanatory information EI output from the generation model GM.

### (Step S14-1)

In step S14-1, the output data generation unit 14 included in the information processing apparatus 100 generates the output data OUT including the second analysis result AR2 and the explanatory information EI.

### (Step S14-2)

In step S14-2, the output data generation unit 14 presents the output data OUT via the input/output unit 40.

### Processing Example 1

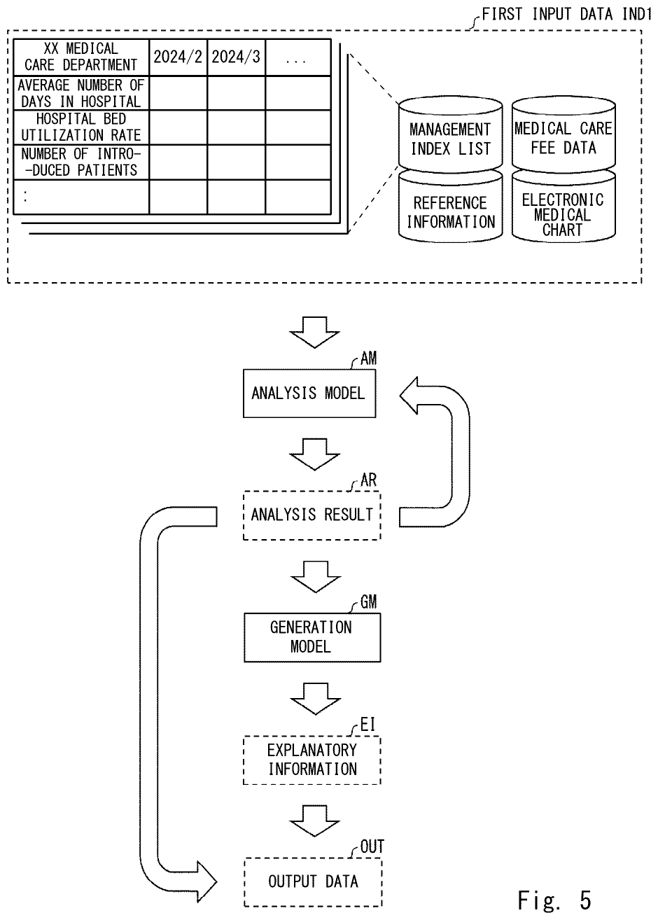

Hereinafter, a specific processing example of the information processing apparatus 100 will be described. FIG. 5 is a diagram showing a processing example of the information processing apparatus 100. In the example in FIG. 5, a flow of data in processing of the information processing apparatus 100 is shown.

As shown in FIG. 5, in the present example, the first input data IND 1 generated by the input data generation unit 121 includes
a management index list of a target medical institution,
data regarding medical care fees at a target medical institution,
data included in an electronic medical chart in a target medical institution, and
other reference information in a target medical institution.
   The management index described above includes, for example,
an average number of days in hospital,
a hospital bed utilization rate, and
the number of introduced patients.

Here, as shown in FIG. 5, as an example, the information processing apparatus 100 may input the first input data INDI to the analysis model AM, and then may repeatedly execute
acquiring the analysis result AR from the analysis model AM,
executing post-processing (post-analysis) on the analysis result AR,
regenerating input data on the basis of post-processing, and
inputting the regenerated input data to the analysis model AM and acquiring the analysis result AR again. The accuracy of the analysis on the target index may be improved by executing the above repetitive processing.

In the post-processing (post-analysis), as an example, presentation of data to the user and reception of an input from the user may be performed, and data to be input to the analysis model AM may be generated by reflecting the input from the user.

As an example, the information processing apparatus 100 may subsequently execute, on the basis of the analysis result AR acquired from the analysis model AM,
generating a prompt with reference to the analysis result AR,
inputting the generated prompt to the generation model GM to acquire the explanatory information EI, and
generating the output data OUT including the analysis result AR and the explanatory information EI.

### (Processing Example of Second Acquisition Unit 12)

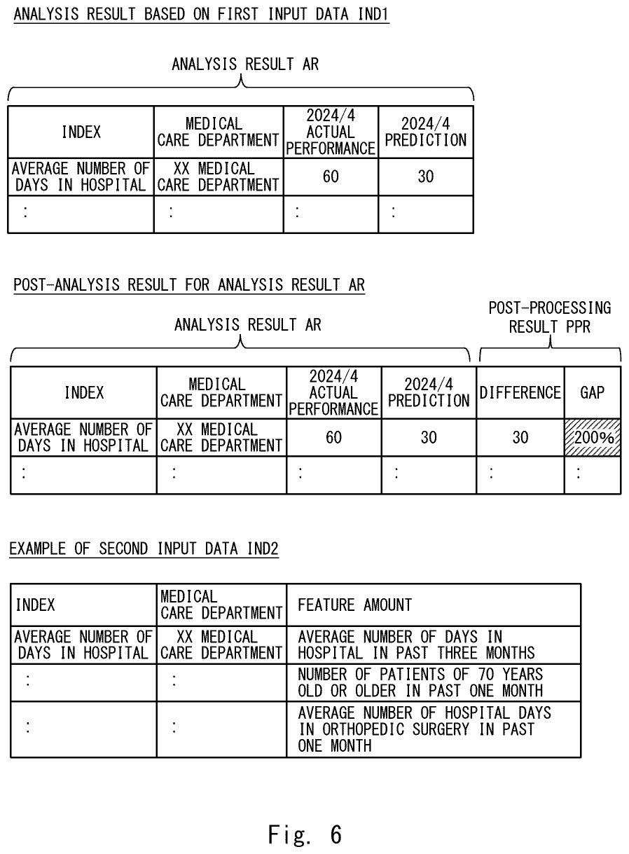

FIG. 6 is a diagram showing a processing example of the second acquisition unit 12. FIG. 6 shows examples of
the analysis result AR based on the first input data IND1,
a post-analysis result for the analysis result AR, and
the second input data IND2.

In the example shown in the upper part of FIG. 6, an “analysis result based on the first input data IND1” is shown as the analysis result AR in the analysis model AM acquired by the second acquisition unit 12. As shown in the upper part of FIG. 6, the “analysis result based on the first input data IND1” includes, for example,
an “average number of hospital days” in “XX medical care department” as the target index TI,
an actual performance value for April 2024, “60”, and
a prediction value of May 2024, “30” as a prediction value predicted by the analysis model AM. As described above, the analysis result in the analysis model AM may include future prediction of the target index TI or the factor index RI.

In the example shown in the middle part of FIG. 6, the input data generation unit 121 included in the second acquisition unit 12 applies post-processing (post-analysis) to the “analysis result based on the first input data IND1”. In the example shown in the middle part of FIG. 6, the input data generation unit 121 calculates a difference between “actual performance” and “prediction” included in the “analysis result based on the first input data IND 1” as the post-processing (post-analysis). “200%” is calculated as a value indicating the deviation (GAP) between the “actual performance” and the “prediction”.

As an example, the input data generation unit 121 applies similar post-processing (post-analysis) to the other target indexes TI included in the “analysis result based on the first input data IND1”, and calculates the deviation between the “actual performance” and the “prediction” for each target index. The input data generation unit 121 specifies an index in which the deviation is relatively large or an index in which the deviation is larger than a predetermined threshold as an index to be further analyzed.

The lower part of FIG. 6 shows an example in which the input data generation unit 121 generates the input data IND2 including a plurality of candidates for the factor index RI that can be a factor of the target index TI with respect to the “average number of days in hospital” which is the target index TI specified in such a manner. As an example, as shown in the lower part of FIG. 6, the input data generation unit 121 generates, as candidates for the factor index RI, the second input data IND2 including feature amounts such as
an average number of days in hospital in past three months,
the number of patients of 70 years old or older in past one month, and
an average number of hospital days in orthopedic surgery in past one month,
and supplies the second input data IND2 to the analysis model AM again. Note that such a candidate for the factor index RI may be selected by the input data generation unit 121 with reference to the past analysis result AR in the analysis model AM as an example. However, the examples are not intended to limit the present exemplary embodiments.

The analysis model AM generates, as an analysis result of the second input data IND2, the analysis result AR including the following information, and analysis result acquisition unit 122 acquires the analysis result AR.
Information indicating a correlation between the “average number of hospital days” in “XX medical care department”, which is the target index TI and the “average number of days in hospital in past three months” which is a candidate for the factor index RI
Information indicating a correlation between the target index TI and “the number of patients of 70 years old or older in past one month” which is a candidate for the factor index RI
Information indicating a correlation between the target index TI and the “average number of hospital days in orthopedic surgery in past one month” which is a candidate for the factor index RI.
   Note that the term “candidate for factor index RI” described above does not limit the present exemplary embodiment, and may be simply expressed as “factor index RI”.

### (Processing Example of Third Acquisition Unit 13)

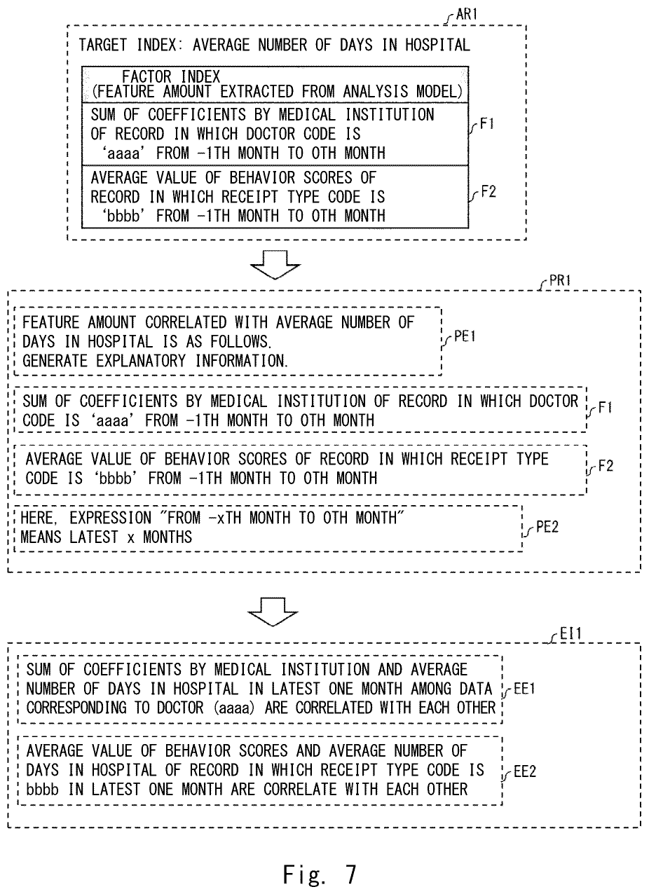

FIG. 7 is a diagram showing a processing example of the third acquisition unit 13. In the example of FIG. 7, as shown in the upper part of FIG. 7, the analysis result AR1 acquired by the second acquisition unit 12 includes
as the target index TI,
the average number of days in hospital”, and includes
as the factor index RI for the target index TI,
a “sum of coefficients by a medical institution of a record in which a doctor code is ‘aaaa’ from −1th month to 0th month”, and
an “average value of behavior scores of a record in which a receipt type code is ‘bbbb’ from −1th month to 0th month”.

As shown in the middle part of FIG. 7, the prompt generation unit 131 included in the third acquisition unit 13 generates a prompt PR1 with reference to the analysis result AR1. Here, the prompt PR1 includes
instruction information PE1,
pieces of feature amount information F1 and F2, and
additional information PE2.
   The instruction information PEI indicates content to be executed by the generation model GM, and in the example shown in the middle part of FIG. 7, the instruction information PE1 includes
a feature amount correlated with the “average number of days in hospital” which is the target index TI is as follows, and
an instruction to generate explanatory information.
   The pieces of feature amount information F1 and F2 are factor indexes RI included in the analysis result ARI described above.

The additional information PE2 indicates items to be referred to by the generation model GM if generating the explanatory information, and in the example shown in the middle part of FIG. 7, the additional information PE2 includes
the expression “from −xth month to 0th month” included in the feature amounts F1 and F2 (factor indexes RI) means the latest x months. Here, the additional information PE2 may be different for each factor index RI. Therefore, the prompt generation unit 131 may be configured to:
generate in advance a prompt template including additional information regarding the factor index RI for each factor index RI, and
select and use a prompt template corresponding to the factor index RI included in the analysis result AR.

The prompt PR1 generated as described above is input to the generation model GM via the communication unit 30. The generation model GM generates explanatory information EI1 as shown in the lower part of FIG. 7 as an example, and the explanatory information acquisition unit 132 acquires the explanatory information EI1. In the example shown in the lower part of FIG. 7, the explanatory information EI1 includes
that the sum of the coefficients by medical institution and the average number of days in hospital in latest one month among the data corresponding to the doctor (aaaa) are correlated with each other (element EE1), and
that the average value of the behavior scores and the average number of days in hospital of the record in which the receipt type code is bbbb in latest one month are correlate with each other (element EE2).

### (Processing Example of Output Data Generation Unit 14)

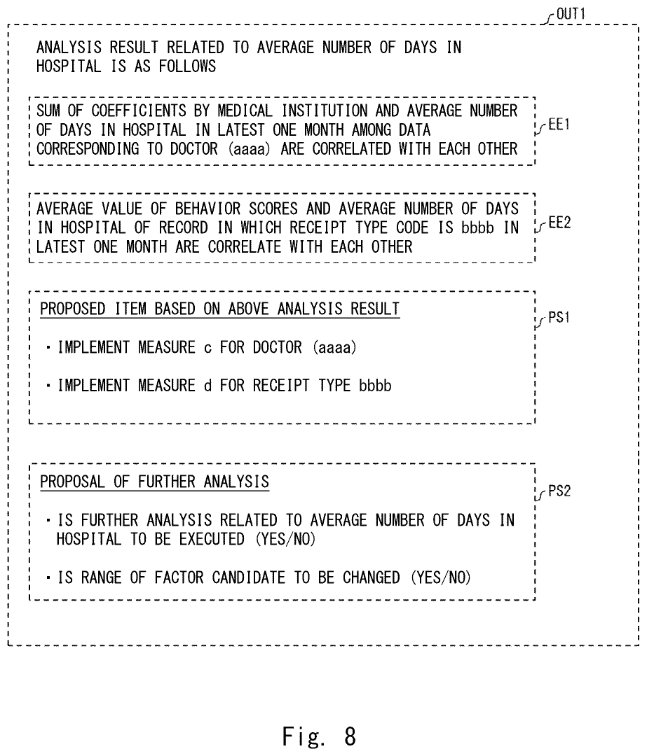

FIG. 8 shows a processing example of the output data generation unit 14. In the example shown in FIG. 8, the output data OUT1 generated by the output data generation unit 14 includes the elements EE1 and EE2 generated by the generation model GM as the explanatory information EI. As shown in FIG. 8, the output data OUT1 may include, as a proposed item PSI to the user based on the analysis result AR1, for example,
implementing a measure c for the doctor (aaaa), and
implementing a measure d for the receipt type bbbb.
   Some of these proposed items may be generated by the generation model GM as an example, or may be generated by the output data generation unit 14 with reference to correspondence information in which an analysis result and a proposed item are associated with each other. Note that these proposed items are examples of information for supporting decision-making regarding management of a target medical institution.

As shown in FIG. 8, the output data OUT1 may include a proposal for prompting the user to perform further analysis. In the case of such a configuration, the first acquisition unit 11 to the third acquisition unit 13 described above may be configured to, on the basis of on answers from the user, execute processes such as
reacquiring the index information IND
regenerating input data and reacquiring the analysis result AR, and
regenerating the prompt PR and reacquiring the explanatory information EI, and
the output data generation unit 14 may be configured to regenerate the output data OUT on the basis of the reacquired explanatory information EI and present the output data OUT to the user.

Note that processing of the output data generation unit 14 is not limited to the above-described example. As an example, the information processing apparatus 100 causes any one of the first acquisition unit 11 to the third acquisition unit 13 to
acquire coping item information indicating an item that can be handled for each medical institution and store the coping item information in the storage unit 20, and
delete a proposed item related to an item difficult to handle in the medical institution through filtering with reference to the coping item information in a case where the output data generation unit 14 generates the output data OUT, and generate the output data OUT including a proposed item related to the item that can be handled by the medical institution.

### Processing Example 2

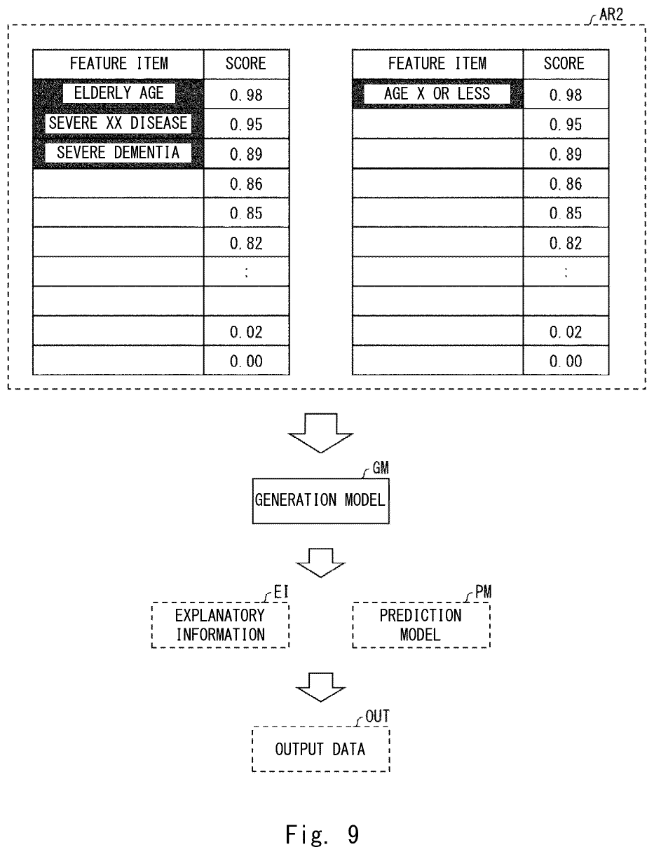

Next, another processing example of the information processing apparatus 100 will be described with reference to FIG. 9. The present processing example may be executed in combination with Processing Example 1 described above, or may be executed separately from Processing Example 1 described above. In the example shown in FIG. 9, the analysis result acquisition unit 122 acquires, from the analysis result AR2, as the factor index RI for a certain target index TI1, feature amounts (“Feature item” shown in FIG. 9) such as
elderly age,
severe XX disease, and
severe dementia, and acquires, as the factor index RI for another target index TI2, a feature amount (“feature item” shown in FIG. 9) such as
age X or less. In the example shown in FIG. 9, the analysis result AR2 includes each score (information regarding correlation) such as
a score indicating a correlation between the target index TI1 and the factor index “elderly age”: 0.98,
a score indicating a correlation between the target index TI1 and the factor index “severe XX disease”: 0.95, and
a score indicating a correlation between the target index TI1 and the factor index “severe dementia”: 0.89.
   Similarly, the analysis result AR2 includes each score (information regarding correlation) such as
a score indicating a correlation between the target index TI2 and the factor index “age X or less”: 0.98.

In the present example, the prompt generation unit 131 generates a prompt PR2 including the analysis result AR2 and including
an instruction to generate the explanatory information RI for explaining the analysis result AR2, and
an instruction to generate a prediction model on the basis of the analysis result AR2,
and inputs the generated prompt PR2 to the generation model GM.

The generation model GM according to the present example generates the explanatory information RI for explaining the analysis result AR2, and generates the prediction model PM on the basis of the analysis result AR2. Here, the specific configuration of the prediction model PM is not limited to the present example, but the prediction model PM is, for example, a machine learning model trained with the analysis result AR2 as learning data. Examples of such a machine learning model may include a model using a neural network or a regression model.

The output data generation unit 14 according to the present example generates output data OUT2 including at least one of.
the explanatory information EI generated by the generation model GM, and
the prediction model PM
and outputs the generated output data OUT2 via the communication unit 30 or the input/output unit 40. In the above example, the case where the generation model GM generates the prediction model PM has been described as an example. However, this does not limit the present exemplary embodiment, and the analysis model AM may generate the prediction model PM.

### (Additional Notes Related to Index Information IND)

The index information IND that is a processing target of the information processing apparatus 100 according to the present exemplary embodiment may include a plurality of indexes in addition to the above-described indexes. Hereinafter, examples of such indexes will be given, but these examples are also not intended to limit the present exemplary embodiment.

### Indexes Related to Hospitalization

a 1 Number of medical care days (day)
a2 Number of new patients (person/day)
a3 Number of discharged patients (person/day)
a4 Number of patients per month (person/month)
a5 Number of patients per hospital period
a6 Number of patients per day (person/day)
a7 Average number of days in hospital (all patients average)
a8 Average number of days in hospital (average for each department)
a9 Hospital bed utilization rate (%)
a10 Medical care unit price (yen/person/day)
a11 Various cost ratios in hospitalization (%)
a12 Medical care fee claim amount
a13 Medical care unit price (yen/person/day)

### Indexes Related to Outpatient

b1 Number of medical care days (day)
b2 Total number of patients (person/month)
b3 Number of patients per day (person/day)
b4 Ratio of new patient (%)
b5 Number of new visits (person/month)
b6 Medical care unit price (yen/person/day)
b7 Various cost ratio in outpatient (%)
b8 Medical care fee claim amount
b9 Medical care unit price (yen/person/day)

### Indexes Common to Hospitalization/Outpatient

c1 Number of introduced patients (person/month)
c2 Introduction ratio (%)
c3 Reverse introduction ratio (%)
c4 Number of new patients (person/month)
c5 Number of surgeries (case)
c5-1 Gastrointestinal endoscopic surgery (case)
c5-2 Endoscopic examination (case)
c5-3 Catheter surgery (case)
c5-4 Radiotherapy cases
c6 Number of CT procedures (case)
c6-1 (Hospitalization) CT procedures (case)
c6-2 (Outpatient) CT procedures (case)
c7 Number of cases where PET-CT was performed (case)
c7-1 (Hospitalization) number of cases where PET-CT was performed (case)
c7-2 (Outpatient) number of cases where PET-CT was performed (case)

Note that, as described above, the factor index RI according to the present exemplary embodiment may include at least one of doctor information, time information, hospitalization information, outpatient information, disease information, treatment information, patient information, and medical care information. Here, as an example, the doctor information may include information associated with each doctor in each item described above. For example, doctor information regarding a doctor A may include, among the items described above, the “a8 average number of days in hospital (average for each department)” or the like described above in which the population is patients in which the doctor A is an attending doctor.

As an example, the time information may include information regarding time in each item described above. For example, “a1 number of medical care days (day)”, “a7 average number of days in hospital (all patient average)”, “a8 average number of days in hospital (average for each department)”, “b1 number of medical care days (day)”, and “a5 number of patients per hospital period” are examples of the time information. The hospitalization information may include at least one of the above-described (indexes related to hospitalization) and (indexes common to hospitalization/outpatient) as an example. The outpatient information may include, as an example, at least one of the above-described (indexes related to outpatient) and (indexes common to hospitalization and outpatient).

The disease information may include information regarding what kind of disease each patient has. The disease information may include information regarding a specific disease such as “c5-4 radiotherapy cases” and “c7 number of cases where PET-CT was performed” among the above-described items.

The treatment information may include information regarding what kind of treatment each patient has received (is scheduled to receive). The treatment information may include information related to specific treatment such as “c5-4 radiotherapy cases” and “c7 number of cases where PET-CT was performed” among the above-described items.

The patient information may include information regarding each patient. As an example, patient information regarding a patient B may include information regarding what kind of disease the patient B has and what kind of treatment the patient B has received (is scheduled to receive). Among the above-described items, the patient information includes “a12 Medical care fee claim amount” and “a13 medical care unit price” related to the patient (yen/person/day)”.

The medical care information may include information regarding medical care of each patient. As an example, the medical care information may include, among the above-described items, “a1 number of medical care days (day)”, “a12 medical care fee claim amount”, “a13 medical care unit price (yen/person/day)”, and the like.

As described above, one or a plurality of target indexes TI according to the present exemplary embodiment include at least one of
an index related to patient admission and discharge,
an index related to a status of a hospital bed, and
an index related to cost incurred with medical service for a patient.
   Here, the above-described “index related to patient admittance and discharge” may include, as an example, at least one of the above-described (indexes related to hospitalization) and (indexes common to hospitalization/outpatient). The “index related to the status of the hospital bed” may include “a9 hospital bed utilization rate (%)” among the above-described items, as an example. The “index related to cost incurred with medical service for a patient” may include an index related to the cost among the above-described items. For example, “a12 medical care fee claim amount”, and “a13 medical care unit price (yen/person/day)” are examples of the “index related to cost incurred with medical service for a patient” described above.

### (Effects of Information Processing System 1A)

As described above, the information processing system 1A is configured to
acquire index information including a plurality of indexes related to a target medical institution,
acquire an analysis result output by an analysis model that has referred to at least a part of the index information,
acquire explanatory information regarding at least a part of the analysis result, the explanatory information being output by a generation model that has been subjected to machine learning and has referred to at least a part of the analysis result, and
generate output data including at least a part of the analysis result and at least a part of the explanatory information.
   As described above, since the output data generated by the information processing system 1A includes at least a part of the analysis result and at least a part of the explanatory information, it is possible to present the analysis result of the index related to the target medical institution to the user in an easily understandable manner.

### [Implementation Example Using Software]

Some or all of the functions of the information processing apparatuses 1 and 100 (hereinafter, also referred to as “each of the above apparatuses”) may be realized by hardware such as an integrated circuit (IC chip) or may be realized by software.

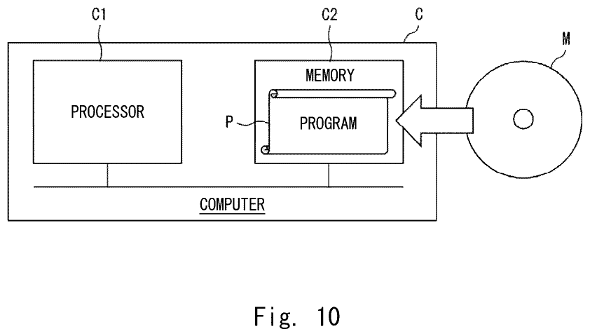

In the latter case, each of the above-described apparatuses is realized by, for example, a computer that executes instructions of a program that is software for realizing each function. An example of such a computer (hereinafter, referred to as a computer C) is shown in FIG. 10. FIG. 10 is a block diagram showing a hardware configuration of the computer C functioning as each of the above apparatuses.

The computer C includes at least one processor C1 and at least one memory C2. A program P for operating the computer C as each of the above apparatuses is recorded in the memory C2. In the computer C, the processor C1 reads the program P from the memory C2 and executes the program P to realize each function of each of the above-described apparatuses.

As the processor C1, for example, a central processing unit (CPU), a graphic processing unit (GPU), a digital signal processor (DSP), a micro processing unit (MPU), a floating point number processing unit (FPU), a physics processing unit (PPU), a tensor processing unit (TPU), a quantum processor, a microcontroller, or a combination thereof may be used. As the memory C2, for example, a flash memory, a hard disk drive (HDD), a solid state drive (SSD), or a combination thereof may be used.

The computer C may further include a random access memory (RAM) for loading the program P at the time of execution and temporarily storing various data. The computer C may further include a communication interface for transmitting and receiving data to and from another apparatus. The computer C may further include an input/output interface for connecting input/output apparatuses such as a keyboard, a mouse, a display, and a printer.

The program P can be recorded on a non-transitory tangible recording medium M readable by the computer C. As such a recording medium M, for example, a tape, a disk, a card, a semiconductor memory, or a programmable logic circuit may be used. The computer C can acquire the program P via such a recording medium M. The program P can be transmitted via a transmission medium. As such a transmission medium, for example, a communication network or a broadcast wave may be used. The computer C can also acquire the program P via such a transmission medium.

Each of the above-described functions of each of the above-described apparatuses may be realized by a single processor provided in a single computer, may be realized by cooperation of a plurality of processors provided in a single computer, or may be realized by cooperation of a plurality of processors provided in each of a plurality of computers. The program for causing each of the above-described apparatuses to realize each of the above-described functions may be stored in a single memory provided in a single computer, may be stored in a distributed manner in a plurality of memories provided in a single computer, or may be stored in a distributed manner in a plurality of memories provided in each of a plurality of computers.

### Additional Note A

The present disclosure includes the techniques described in the following supplementary notes. However, the present disclosure is not limited to the techniques described in the following Supplementary Notes, and various modifications can be made within the scope described in the claims.

### (Supplementary Note A1)

An information processing apparatus including:
first acquisition means for acquiring index information including a plurality of indexes related to a target medical institution;
second acquisition means for acquiring an analysis result output by an analysis model that has referred to at least a part of the index information;
third acquisition means for acquiring explanatory information regarding at least a part of the analysis result, the explanatory information being information output from a generation model that has been subjected to machine learning and has referred to at least a part of the analysis result; and
generation means for generating output data including at least a part of the analysis result and at least a part of the explanatory information.

### (Supplementary Note A2)

The information processing apparatus according to Supplementary Note A1, in which the analysis result includes information regarding a correlation between one or a plurality of target indexes obtained from the index information and one or a plurality of factor indexes obtained from the index information.

### (Supplementary Note A3)

The information processing apparatus according to Supplementary Note A2, in which the third acquisition means includes prompt generation means for generating a prompt to be input to the generation model with reference to at least a part of the analysis result.

### (Supplementary Note A4)

The information processing apparatus according to Supplementary Note A3, in which the prompt generation means generates a prompt to be input to the generation model by using a prompt template preset for each factor index.

### (Supplementary Note A5)

The information processing apparatus according to any one of Supplementary Notes A2 to A4, in which the one or plurality of factor indexes are indexes related to at least one of doctor information, time information, hospitalization information, outpatient information, disease information, treatment information, patient information, and medical care information included in the index information.

### (Supplementary Note A6)

The information processing apparatus according to any one of Supplementary Notes A2 to A4,
in which the one or plurality of target indexes include at least one of an index related to patient admission and discharge,
an index related to a status of a hospital bed, and
an index related to cost incurred with a medical service for a patient.

### (Supplementary Note A7)

The information processing apparatus according to Supplementary Note A6, in which the one or plurality of target indexes include at least one of an average number of days in hospital, a hospital bed utilization rate, and the number of introduced patients.

### (Supplementary Note A8)

The information processing apparatus according to Supplementary Note A7, in which the output data includes information for supporting decision-making regarding management of the target medical institution.

### Additional Note B

The present disclosure includes the techniques described in the following supplementary notes. However, the present disclosure is not limited to the techniques described in the following Supplementary Notes, and various modifications can be made within the scope described in the claims.

### (Supplementary Note B1)

An information processing method including:
a first acquisition process in which at least one processor acquires index information including a plurality of indexes related to a target medical institution;
a second acquisition process in which the at least one processor acquires an analysis result output by an analysis model that has referred to at least a part of the index information;
a third acquisition process in which the at least one processor acquires explanatory information regarding at least a part of the analysis result, the explanatory information being information output from a generation model that has been subjected to machine learning and has referred to at least a part of the analysis result; and
a generation process in which the at least one processor generates output data including at least a part of the analysis result and at least a part of the explanatory information.

### (Supplementary Note B2)

The information processing method according to Supplementary Note B1, in which the analysis result includes information regarding a correlation between one or a plurality of target indexes obtained from the index information and one or a plurality of factor indexes obtained from the index information.

### (Supplementary Note B3)

The information processing method according to Supplementary Note B2, in which the third acquisition process includes a prompt generation process in which the at least one processor generates a prompt to be input to the generation model with reference to at least a part of the analysis result.

### (Supplementary Note B4)

The information processing method according to Supplementary Note B3, in which, in the prompt generation process, the at least one processor generates a prompt to be input to the generation model by using a prompt template preset for each factor index.

### (Supplementary Note B5)

The information processing method according to any one of Supplementary Notes B2 to B4, in which the one or plurality of factor indexes are indexes related to at least one of doctor information, time information, hospitalization information, outpatient information, disease information, treatment information, patient information, and medical care information included in the index information.

### (Supplementary Note B6)

The information processing method according to any one of Supplementary Notes B2 to B4,
in which the one or plurality of target indexes include at least one of
an index related to patient admission and discharge,
an index related to a status of a hospital bed, and
an index related to cost incurred with a medical service for a patient.

### (Supplementary Note B7)

The information processing method according to Supplementary Note B6, wherein the one or plurality of target indexes include at least one of an average number of days in hospital, a hospital bed utilization rate, and the number of introduced patients.

### (Supplementary Note B8)

The information processing method according to Supplementary Note B7, in which the output data includes information for supporting decision-making regarding management of the target medical institution.

### Additional Note C

The present disclosure includes the techniques described in the following supplementary notes. However, the present disclosure is not limited to the techniques described in the following Supplementary Notes, and various modifications can be made within the scope described in the claims.

### (Supplementary Note C1)

An information processing program for causing a computer to function as an information processing apparatus and causing the computer to function as:
first acquisition means for acquiring index information including a plurality of indexes related to a target medical institution;
second acquisition means for acquiring an analysis result output by an analysis model that has referred to at least a part of the index information;
third acquisition means for acquiring explanatory information regarding at least a part of the analysis result, the explanatory information being information output from a generation model that has been subjected to machine learning and has referred to at least a part of the analysis result; and
generation means for generating output data including at least a part of the analysis result and at least a part of the explanatory information.

### (Supplementary Note C2)

The information processing program according to Supplementary Note C1, in which the analysis result includes information regarding a correlation between one or a plurality of target indexes obtained from the index information and one or a plurality of factor indexes obtained from the index information.

### (Supplementary Note C3)

The information processing program according to Supplementary Note C2, in which the computer further functions as prompt generation means of generating a prompt to be input to the generation model with reference to at least a part of the analysis result.

### (Supplementary Note C4)

The information processing program according to Supplementary Note C3, in which the prompt generation means generates a prompt to be input to the generation model by using a prompt template preset for each factor index.

### (Supplementary Note C5)

The information processing program according to any one of Supplementary Notes C2 to C4, in which the one or plurality of factor indexes are indexes related to at least one of doctor information, time information, hospitalization information, outpatient information, disease information, treatment information, patient information, and medical care information included in the index information.

### (Supplementary Note C6)

The information processing program according to any one of Supplementary Notes C2 to C4,
in which the one or plurality of target indexes include at least one of
an index related to patient admission and discharge,
an index related to a status of a hospital bed, and
an index related to cost incurred with a medical service for a patient.

### (Supplementary Note C7)

The information processing program according to Supplementary Note C6, in which the one or plurality of target indexes include at least one of an average number of days in hospital, a hospital bed utilization rate, and the number of introduced patients.

### (Supplementary Note C8)

The information processing program according to Supplementary Note C7, in which the output data includes information for supporting decision-making regarding management of the target medical institution.

### Additional Note D

The present disclosure includes the techniques described in the following supplementary notes. However, the present disclosure is not limited to the techniques described in the following Supplementary Notes, and various modifications can be made within the scope described in the claims.

### (Supplementary Note D1)

An information processing apparatus including at least one processor,
in which the at least one processor executes
a first acquisition process of acquiring index information including a plurality of indexes related to a target medical institution,
a second acquisition process of acquiring an analysis result output by an analysis model that has referred to at least a part of the index information,
a third acquisition process of acquiring explanatory information regarding at least a part of the analysis result, the explanatory information being information output from a generation model that has been subjected to machine learning and has referred to at least a part of the analysis result, and
a generation process of generating output data including at least a part of the analysis result and at least a part of the explanatory information.

The information processing apparatus may further include a memory. The memory may store a program for causing the at least one processor to execute each process.

### (Supplementary Note D2)

The information processing apparatus according to Supplementary Note D1, in which the analysis result includes information regarding a correlation between one or a plurality of target indexes obtained from the index information and one or a plurality of factor indexes obtained from the index information.

### (Supplementary Note D3)

The information processing apparatus according to Supplementary Note D2, in which the at least one processor further executes a prompt generation process of generating a prompt to be input to the generation model with reference to at least a part of the analysis result.

### (Supplementary Note D4)

The information processing apparatus according to Supplementary Note D3, in which, in the prompt generation process, the at least one processor is configured to generate a prompt to be input to the generation model by using a prompt template preset for each factor index.

### (Supplementary Note D5)

The information processing apparatus according to any one of Supplementary Notes D2 to D4, in which the one or plurality of factor indexes are indexes related to at least one of doctor information, time information, hospitalization information, outpatient information, disease information, treatment information, patient information, and medical care information included in the index information.

### (Supplementary Note D6)

The information processing apparatus according to any one of Supplementary Notes D2 to D4,
in which the one or plurality of target indexes include at least one of
an index related to patient admission and discharge,
an index related to a status of a hospital bed, and
an index related to cost incurred with a medical service for a patient.

### (Supplementary Note D7)

The information processing apparatus according to Supplementary Note D6, in which the one or plurality of target indexes include at least one of an average number of days in hospital, a hospital bed utilization rate, or the number of introduced patients.

### (Supplementary Note D8)

The information processing apparatus according to Supplementary Note D7, in which the output data includes information for supporting decision-making regarding management of the target medical institution.

### Additional Note E

The present disclosure includes the techniques described in the following supplementary notes. However, the present disclosure is not limited to the techniques described in the following Supplementary Notes, and various modifications can be made within the scope described in the claims.

### (Supplementary Note E1)

A non-transitory computer readable medium recording an information processing program for causing a computer to function as an information processing apparatus, the program causing the computer to execute:
a first acquisition process of acquiring index information including a plurality of indexes related to a target medical institution;
a second acquisition process of acquiring an analysis result output by an analysis model that has referred to at least a part of the index information;
a third acquisition process of acquiring explanatory information regarding at least a part of the analysis result, the explanatory information being information output from a generation model that has been subjected to machine learning and has referred to at least a part of the analysis result; and
a generation process of generating output data including at least a part of the analysis result and at least a part of the explanatory information.

While the present disclosure has been particularly shown and described with reference to example embodiments thereof, the present disclosure is not limited to these example embodiments. It will be understood by those of ordinary skill in the art that various changes in form and details may be made therein without departing from the sprit and scope of the present disclosure as defined by the claims. And each embodiment can be appropriately combined with at least one of embodiments.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

What is claimed is:

1. An information processing apparatus comprising:
at least one memory storing instructions; and
at least one processor configured to execute the instructions to:
acquire index information including a plurality of indexes related to a target medical institution;
acquire an analysis result output by an analysis model that has referred to at least a part of the index information;
acquire explanatory information regarding at least a part of the analysis result, the explanatory information being information output from a generation model that has been subjected to machine learning and has referred to at least a part of the analysis result; and
generate output data including at least a part of the analysis result and at least a part of the explanatory information.

2. The information processing apparatus according to claim 1, wherein the analysis result includes information regarding a correlation between one or a plurality of target indexes obtained from the index information and one or a plurality of factor indexes obtained from the index information.

3. The information processing apparatus according to claim 2, wherein the at least one processor is further configured to execute the instructions to generate a prompt to be input to the generation model with reference to at least a part of the analysis result.

4. The information processing apparatus according to claim 3, wherein the at least one processor is further configured to execute the instructions to generate a prompt to be input to the generation model by using a prompt template preset for each factor index.

5. The information processing apparatus according to claim 2, wherein the one or plurality of factor indexes are indexes related to at least one of doctor information, time information, hospitalization information, outpatient information, disease information, treatment information, patient information, and medical care information included in the index information.

6. The information processing apparatus according to claim 2,
wherein the one or plurality of target indexes include at least one of
an index related to patient admission and discharge,
an index related to a status of a hospital bed, and
an index related to cost incurred with a medical service for a patient.

7. The information processing apparatus according to claim 6, wherein the one or plurality of target indexes include at least one of an average number of days in hospital, a hospital bed utilization rate, and the number of introduced patients.

8. The information processing apparatus according to claim 7, wherein the output data includes information for supporting decision-making regarding management of the target medical institution.

9. An information processing method comprising:
acquiring index information including a plurality of indexes related to a target medical institution;
acquiring an analysis result output by an analysis model that has referred to at least a part of the index information;
acquiring explanatory information regarding at least a part of the analysis result, the explanatory information being information output from a generation model that has been subjected to machine learning and has referred to at least a part of the analysis result; and
generating output data including at least a part of the analysis result and at least a part of the explanatory information.

10. A non-transitory computer readable medium storing a program causing a computer to function as an information processing apparatus, the program causing the computer to execute:
acquiring index information including a plurality of indexes related to a target medical institution;
acquiring an analysis result output by an analysis model that has referred to at least a part of the index information;
acquiring explanatory information regarding at least a part of the analysis result, the explanatory information being information output from a generation model that has been subjected to machine learning and has referred to at least a part of the analysis result; and
generating output data including at least a part of the analysis result and at least a part of the explanatory information.
